SEQUENCE LISTING (34 sequences)

SEQ ID NO: 1 - protein, 6 aa, synthetic construct
UUUUUU

SEQ ID NO: 2 - protein, 64 aa, synthetic construct
GAUUUAGACUACCCCAAAAACGAAGGGGACUAAAACGGGCGCGUUCAGGGUGGUAUGGCCGUAG

SEQ ID NO: 3 - protein, 58 aa, synthetic construct
GACCACCCCAAAAAUGAAGGGGACUAAAACGGGCGCGUUCAGGGUGGUAUGGCCGUAG

SEQ ID NO: 4 - protein, 58 aa, synthetic construct
GACCACCCCAAAAAUGAAGGGGACUAAAACUAGACUAGUGGCAACCAAAACGGCCGGA

SEQ ID NO: 5 - protein, 58 aa, synthetic construct
GACCACCCCAAAAAUGAAGGGGACUAAAACATCACTCACAGAGTGACATCGAAAAATA

SEQ ID NO: 6 - protein, 58 aa, synthetic construct
GACCACCCCAAAAAUGAAGGGGACUAAAACAGAUAAUCACUCACAGAGUGACAUCGAA

SEQ ID NO: 7 - protein, 50 aa, synthetic construct
GACCACCCCAAAAAUGAAGGGGACUAAAACACATCGAAAAATACCCTTGT

SEQ ID NO: 8 - protein, 50 aa, synthetic construct
GACCACCCCAAAAAUGAAGGGGACUAAAACACAGAGTGACATCGAAAAAT

SEQ ID NO: 9 - protein, 58 aa, synthetic construct
GACCACCCCAAAAAUGAAGGGGACUAAAACAGAGTGACATCGAAAAATACCCTTGTTT

SEQ ID NO: 10 - protein, 58 aa, synthetic construct
GACCACCCCAAAAAUGAAGGGGACUAAAACACAGAGTGACATCGAAAAATACCCTTGT

SEQ ID NO: 11 - protein, 58 aa, synthetic construct
GACCACCCCAAAAAUGAAGGGGACUAAAACAGTGACATCGAAAAATACCCTTGTTTCT

SEQ ID NO: 12 - protein, 50 aa, synthetic construct
GACCACCCCAAAAAUGAAGGGGACUAAAACCAGAGTGACATCGAAAAATA

SEQ ID NO: 13 - protein, 50 aa, synthetic construct
GACCACCCCAAAAAUGAAGGGGACUAAAACTGACATCGAAAAATACCCTT

SEQ ID NO: 14 - protein, 58 aa, synthetic construct
GACCACCCCAAAAAUGAAGGGGACUAAAACTAGTTACCCTGCTTTTGCTGCCACTAGT

SEQ ID NO: 15 - protein, 58 aa, synthetic construct
GACCACCCCAAAAAUGAAGGGGACUAAAACTAGTATGGGTGCTTTTGCTGCCACTAGT

SEQ ID NO: 16 - protein, 71 aa, synthetic construct
AGUAGAAACAAGGGUAUUUUUCUUUACUAGUGGCAGCAAAAGCAGGGUAACUAGUCUACCCUGCUUUUGCU

SEQ ID NO: 17 - protein, 71 aa, synthetic construct
AGUAGAAACAAGGGUAUUUUUCUUUACUAGUGGCAGCAAAAGCACCCAUACUAGUCUACCCUGCUUUUGCU

SEQ ID NO: 18 - protein, 71 aa, synthetic construct
AGUAGAAACAAGGGUAUUUUUCUUUACUAGUCCGGCCGUUUUGGUUGCCACUAGUCUACCCUGCUUUUGCU

SEQ ID NO: 19 - protein, 61 aa, synthetic construct
AGUAGAAACAAGGGUAUUUUUCGAUGUCACUCUGUGAGUGAUUAUCUACCCUGCUUUUGCU

SEQ ID NO: 20 - protein, 75 aa, synthetic construct
AGTAGAAACAAGGGCACTACTGGAAAACTACCTGTTCCATGGCCAACACTTGTCACTACTTTCCCTGCTTTTGCT

SEQ ID NO: 21 - protein, 100 aa, synthetic construct
AGTAGAAACAAGGGTAACAGCTGCTGGGATTACACATGGCATGGATGAACTATACAAATAAATGTCCAGACCTGCAGGCATGCAAGCTCCTGCTTTTGCT

SEQ ID NO: 22 - protein, 120 aa, synthetic construct
AGTAGAAACAAGGGCGATGGCCCTGTCCTTTTACCAGACAACCATTACCTGTCCACACAATCTGCCCTTTCGAAAGATCCCAACGAAAAGAGAGACCACATGGTCCTTCCTGCTTTTGCT

SEQ ID NO: 23 - protein, 22 aa, synthetic construct
AGCAAAAGCAGGGTAGACTAGT

SEQ ID NO: 24 - protein, 22 aa, synthetic construct
AGTAGAAACAAGGGTATTTTTC

SEQ ID NO: 25 - protein, 9 aa, synthetic construct
TTACTAGTC

SEQ ID NO: 26 - protein, 16 aa, synthetic construct
CGGCCGTTTTGGTTGC

SEQ ID NO: 27 - protein, 29 aa, synthetic construct
GTTCAGACGTGTGCTCTTCCGATCTAGCG

SEQ ID NO: 28 - protein, 8 aa, synthetic construct
AAAGCAGG

SEQ ID NO: 29 - protein, 28 aa, synthetic construct
GTTCAGACGTGTGCTCTTCCGATCTAGC

SEQ ID NO: 30 - protein, 8 aa, synthetic construct
AAAGCAGG

SEQ ID NO: 31 - protein, 34 aa, synthetic construct
CACGACGCTCTTCCGATCTHNNNNNNNAGTAGAA

SEQ ID NO: 32 - protein, 5 aa, synthetic construct
CAAGG

SEQ ID NO: 33 - protein, 40 aa, synthetic construct
GAGATCTACACTCTTTCCCTACACGACGCTCTTCCGATCT

SEQ ID NO: 34 - protein, 45 aa, synthetic construct
GAGATACTGGTGTGACTGGAGTTCAGACGTGTGCTCTTCCGATCT

(19) United States
(12) Patent Application Publication
(10) Pub. No.: US 2025/0146089 A1
(43) Pub. Date: May 8, 2025

(54) ABERRANT VIRAL RNA DETECTION USING CAS13

(71) Applicant: The Trustees of Princeton University, Princeton, NJ (US)

(72) Inventors: Caitlin Lamb, Princeton, NJ (US); Arend Jan Wouter te Velthuis, Princeton, NJ (US); Cameron Myhrvold, Princeton, NJ (US)

(73) Assignee: The Trustees of Princeton University, Princeton, NJ (US)

(21) Appl. No.: 18/936,289

(22) Filed: Nov. 4, 2024

Related U.S. Application Data
(60) Provisional application No. 63/547,239, filed on Nov. 3, 2023.

(51) Int. Cl. C12Q 1/70; C12N 9/22; C12N 15/11; C12Q 1/6818

(52) U.S. Cl. CPC C12Q 1/701; C12N 9/22; C12N 15/11; C12Q 1/6818; C12N 2310/20

(57) ABSTRACT

Disclosed is a method for quantitative detection of a small, aberrant viral nucleic acid. The method includes combining a detection mixture with a sample, and allowing a reaction with the test mixture to begin. The sample includes an RNA sample. The detection mixture includes a Cas13 enzyme, a reporter, a sequence-specific CRISPR RNA (crRNA), and a buffer. The method includes determining a quantitative value representative of the reporter in the test mixture, where the quantitative value is a measure of an amount of target RNA present in the RNA sample.

### CROSS-REFERENCE TO RELATED APPLICATIONS

The present application claims priority to U.S. Provisional Patent App. No. 63/547,239, filed Nov. 3, 2023, the contents of which are incorporated by reference herein in its entirety.

### STATEMENT REGARDING FEDERALLY SPONSORED RESEARCH OR DEVELOPMENT

This invention was made with government support under Grant Nos. AI168808, UL1TR003017, AI175474, and GM007388 awarded by the National Institutes of Health, and Grant No. 75D30122C15113 awarded by the Centers for Disease Control & Prevention. The government has certain rights in the invention.

### SEQUENCE LISTING

The present application contains a Sequence Listing which has been submitted electronically in XML file format and is hereby incorporated by reference in its entirety. The XML copy, created on Oct. 24, 2024, is titled PRIN-97902_SL.xml and is 31,063 bytes in size.

### TECHNICAL FIELD

The present disclosure is drawn to viral detection techniques for ribonucleic acid (RNA), and specifically, aberrant viral RNA detection techniques.

### BACKGROUND

Many viruses, such as those that infect humans, release viral ribonucleic acid (vRNA) upon infection. During the viral replication and transcription phase, aberrant RNA can be generated. For example, viral RNA-dependent RNA polymerases (RdRps) lack proofreading capability, and during replication this can result in high mutation rates, leading to populations of closely related variant sequences.

Current tools to detect and quantify aberrant RNAs include primer extension, reverse transcription quantitative (RT) polymerase chain reaction (PCR) (RT-qPCR), and next generation sequencing. All RT and PCR enzymes can introduce errors, including the introduction of point mutations, insertions, and deletions, which may impact the detection of viral RNAs and create new molecules that also deviate from the viral genome sequence. Various approaches have been used to minimize these errors, such as circular sequencing. However, a more accurate measurement technique is needed.

### BRIEF SUMMARY

In various aspects, a method for quantitative detection of a small, aberrant viral nucleic acid (which may include synthetic RNA and/or RNA produced by a virus) may be provided. It is envisioned that this may be used for any virus, including, e.g., a coronavirus, an arenavirus, a hantavirus, a paramyxovirus, an Ebola virus, a picornavirus, or a flavivirus.

The method may include forming a test mixture by combining a detection mixture with a sample. The sample may include an RNA sample. The detection mixture may include a Cas13 enzyme, a reporter (such as a fluorescent reporter, a lateral flow reporter, a colorimetric reporter, or a luminescent reporter), a sequence-specific CRISPR RNA (crRNA), and a buffer. The Cas13 enzyme may preferably be LbuCas13a or LwaCas13a, but in some aspects, the Cas13 enzyme may be a Cas13 enzyme other than LbuCas13a or LwaCas13a.

The method may include allowing a reaction within the test mixture to begin. The method may include determining a quantitative value (such as a fluorescence or lateral flow readout) representative of the reporter in the test mixture, where the quantitative value is a measure of an amount of target RNA present in the RNA sample.

The method may be free of an amplification step. In some aspects, the method may include pre-amplification of the RNA sample. The method may include RNA fractionation. In some aspects, the sample may include an antiviral, and the method may include monitoring an infection status by repeating the forming, allowing, and determining steps and tracking the quantitative value over time.

In various aspects, a composition of matter (e.g., a detection mixture) may be provided. The composition of matter may include a Cas13 enzyme, a reporter (such as a quenched fluorescent reporter or an affinity-based reporter), a sequence-specific CRISPR RNA (crRNA), and a buffer. The Cas13 enzyme may preferably be LbuCas13a or LwaCas13a, but in some aspects, the Cas13 enzyme may be a Cas13 enzyme other than LbuCas13a or LwaCas13a. The sequence-specific crRNA may be configured to target a junction sequence formed upon aberrant viral RNA (such as mini viral RNA (mvRNA)) production. In some aspects, the composition of matter may include sample RNA.

In various aspects, a detection system may be provided. The detection system may include a detection mixture container, where a composition of matter as disclosed herein may be disposed within the detection mixture container. The detection system may include a detector configured to detect fluorescence of the composition of matter.

In various aspects, a detection kit may be provided. The detection kit may include a sample container and a detection mixture container. A composition of matter as disclosed herein may be disposed within the detection mixture container.

### BRIEF DESCRIPTION OF THE FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a schematic of influenza A virus (IAV) RNA replication, where the IAV RNA polymerase forms a dimer during replication; this dimer is stabilized by host factor ANP32A/B and able to recruit viral nucleoprotein (NP) to encapsidate nascent viral RNA. In addition to producing full length RNA products, IAV replication also produces mini viral RNAs (mvRNA) and defective viral genomes (DVG).

FIG. 2 is a schematic showing that during IAV replication mvRNAs can be generated through a copy-choice recombination mechanism that deletes internal genome segment sequences; crRNAs can be designed to target the unique junction sequences, thereby distinguishing mvRNAs from full length viral genome segments and other mvRNAs.

FIG. 3 is a flowchart of a method.

FIG. 4 is a schematic illustration of a method.

FIG. 5 is a graph of the detection of 5S rRNA using LbuCas13a or LwaCas13a. Data points indicate technical repeats.

FIG. 6 is a graph Detection synthetic mvRNA NP71.6 diluted in water or HEK293T total RNA using LbuCas13a. Data points indicate technical repeats.

FIG. 7 is a graph showing comparisons of LbuCas13a activity with detection mixtures using a standard SHINE buffer to the LbuCas13a activity in modified detection mixtures having a 2-fold higher crRNA concentration (10 nM), a 14 mM MgOAc to 14 mM MgCl2 substitution, and a 5 mM MgOAc concentration, when used with synthetic mvRNA NP71.6 diluted in water. Data points indicate technical repeats.

FIG. 8 is a graph showing comparisons of LbuCas13a activity with detection mixtures using a standard SHINE buffer to the LbuCas13a activity in modified detection mixtures using TRIS-HCl instead of HEPES buffer, when used with synthetic mvRNA NP71.6 diluted in water. Data points indicate technical repeats.

FIG. 9 is a graph showing comparisons of LbuCas13a activity with detection mixtures using a standard SHINE buffer to the LbuCas13a activity in modified detection mixtures without 5% PEG or with the presence of 5% glycerol, when used with synthetic mvRNA NP71.6 diluted in water. Data points indicate technical repeats.

FIG. 10 is a graph showing detection of synthetic mvRNAs NP71.10 and NP71.11 diluted in HEK293T total RNA using LbuCas13a and crRNAs CL68 and CL69, respectively.

FIG. 11 is a schematic illustration of RNA sample preparation for mvRNA detection.

FIG. 12A is a graph showing LbuCas13a detection of synthetic mvRNA NP71.6 diluted in HEK293T total RNA.

FIG. 12B is a graph showing a close-up of the bottom part of the graph of FIG. 12A.

FIG. 13 is a set of graphs showing detection of chemically synthesized or transfected mvRNA NP71.6 in the presence of wildtype or PBla mutant IAV RNA polymerase that contained alanine substitutions at position D445 and D446 using LbuCas13a (top), TaqMan-based RT-qPCR (middle) or primer extension (bottom).

FIG. 14 is a graph showing Michaelis-Menten fits to the maximum rate of fluorescence as a function of synthetic mvRNA NP71.6 copy number.

FIG. 15 is a bar graph showing copy number of mvRNA NP71.6 in transfection samples. Data points indicate three biological repeats. Statistical comparisons are based on non-parametric t-test. In all graphs, error bars indicate standard deviation.

FIG. 16 is a heatmap showing normalized fluorescence after 180 min of Cas13 detection. 9 different crRNA designs were used, and their ability to detect synthetic NP-61 in standard curve or 5 ng of a T7 segment 5 vRNA transcript in the presence of A549 total RNA was tested. In addition, A549 cells infected with WSN were fractionated into large (>200 nt) and small (<200 nt) RNA fractions. The NP-61 mvRNA was expected to be solely present and detected in the <200 nt fraction. The size of the crRNAs is indicated.

FIG. 17 is a chart showing copy number of myRNA NP-61 in HEK 293T transfection. Data points indicate biological repeats of separate transfections.

FIG. 18 is a chart showing detection of mvRNAs NP-61 in small RNA (<200 nt) fractions extracted from ferret lung or nasal turbinates homogenates infected with influenza A/Brevig Mission/1/1918 (H1N1). Statistical comparisons were performed using One-way ANOVA.

FIG. 19 is a chart showing detection of myRNAs NP-61 in small RNA (<200 nt) fractions extracted from ferret lung homogenates infected with influenza A/Netherlands/2009 (H1N1). Statistical comparisons were performed using One-way ANOVA.

FIG. 20 is a chart showing detection of mvRNAs NP-61 in small RNA (<200 nt) fractions extracted from ferret lung homogenates infected with influenza A/Indonesia/2005 (H5N1). Data points indicate separate biological infections or mock infections. Statistical comparisons were performed using One-way ANOVA.

FIG. 21 is a chart showing copy number of NP-61-like RNAs (mvRNA and DVG) in equal volumes of total RNA extracted from clinical nasopharyngeal samples for the samples with copy number values above the limit of detection.

FIG. 22 is a chart showing copy number of NP-61-like RNAs plotted against the amount of ng total RNA used.

FIG. 23 is a chart showing copy number of NP-61-like mvRNAs in equal volumes of total RNA extracted from clinical nasopharyngeal samples for the samples with copy number values above the limit of detection.

FIGS. 24-27 are charts showing copy number of NP-61-like mvRNAs plotted against (FIG. 24) concentration of <200 nt RNA, (FIG. 25) clinical RT-qPCR Ct value, (FIG. 26) patient age, and (FIG. 27) patient sex. Statistical comparisons are based on non-parametric t-test. Not significant is indicated with n.s.

FIG. 28 is a schematic illustration of an mcRNA transfection protocol.

FIG. 29 are charts showing different mvRNA sequences having varying effects on an IFN response.

FIGS. 30-33 are graphs illustrating that different mvRNAs have different kinetics.

FIG. 34 is a set of graphs showing how PA-50 AND PA-66 localize to different compartments.

FIGS. 35 and 36 are a set of graphs showing a treatment using Baloxavir and adding RdRp allows for replication while inhibiting transcription, for virion-sense (V-sense) (FIG. 35) and complementary-sense (C-sense) (FIG. 36) strands.

FIGS. 37 and 38 are graphs showing detection of a segment from ANDV (FIG. 37) and TULV (FIG. 38)

FIGS. 39 and 40 are graphs showing detection of a large segment (L segment) from ANDV infection.

FIGS. 41 and 42 are graphs showing detection of a large segment (L segment) defective viral genomes (DVGs) from ANDV infection; FIG. 42 is scaled to only show the BN233 and Mock DVG data.

FIGS. 43 and 44 are graphs showing detection of 5′ ends from SARS-COV-2 infected (TC) samples.

FIG. 45 is a graph showing detection of SARS-COV-2 genome and svRNA.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

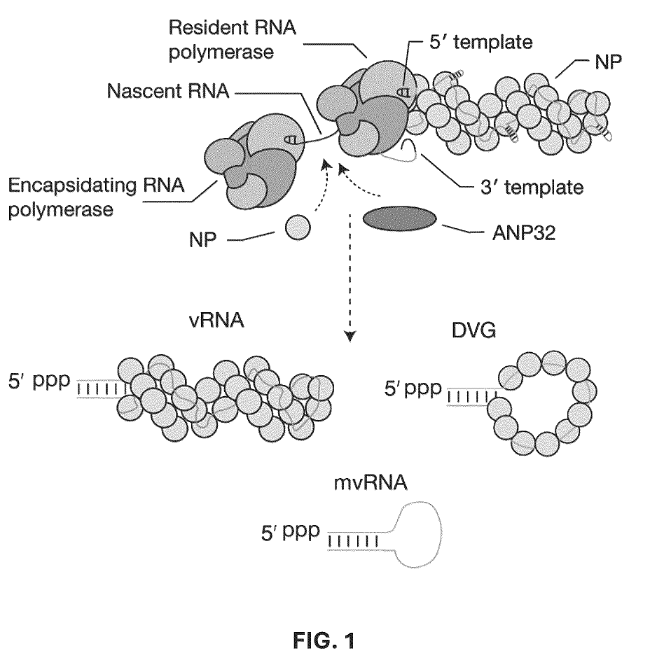

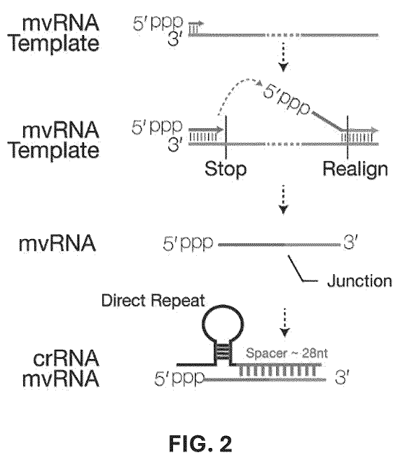

Influenza A viruses (IAV) cause mild to severe respiratory disease in humans, depending on the viral strain and activation of the innate immune response. Upon infection, IAV releases eight segments of negative-sense, single-stranded RNA that are organized into viral nucleoprotein (vRNP) complexes. Each vRNP complex consists of a viral RNA (vRNA) that is bound by a helical coil of nucleoproteins (NP) and a copy of the RNA-dependent viral RNA polymerase. See FIG. 1. During replication, the IAV RNA polymerase can produce a wide variety of aberrant or nonstandard RNA products, including defective viral genomes (DVG) and mini viral RNAs (mvRNA), which contain the conserved termini of the viral genome segments but lack internal sequences. See FIG. 1. It is currently assumed that the internal deletions are the result of an intramolecular copy-choice recombination event that involves pausing of the RNA polymerase at an unknown signal and realignment of the nascent RNA to a complementary sequence downstream. Deletion of an internal sequence, results in the formation of a unique junction sequence relative to the full-length viral genome. See FIG. 2.

Some of the aberrant RNAs have been shown to bind and activate retinoic-acid inducible gene I (RIG-I), leading to downstream innate immune signaling. The production of aberrant RNAs plays a still poorly understood role in viral pathogenicity and the outcome of influenza virus infection. In the case of mvRNAs, their abundance was associated with the appearance of disease markers in mouse and ferret infections with highly pathogenic IAV strains. However, not all aberrant RNA molecules are able to induce an innate immune response. In IAV, as well as other negative sense RNA viruses, sequence-specific preferences for RIG-I binding and activation have been observed. For IAV, it was recently showed that mvRNAs containing a template loop (t-loop), a transient RNA structure that can affect RNA polymerase processivity, are more potent inducers of the innate immune response than mvRNAs without a t-loop in cell culture, although the sensitivity to t-loops is IAV RNA polymerase-dependent. Given the large variety of aberrant RNA sequences produced during IAV infection and the specific effects of different sequences, it is becoming more important to carefully study the kinetics and impact of aberrant RNA species during infection.

The disclosed method can complement current assays and performs aberrant viral RNA detection preferably without RT and PCR steps. The disclosed method utilizes Cas13 enzymes for direct and amplification-free detection of aberrant RNAs; without enzymatically manipulation of the genetic material that needs to be detected, a more accurate measurement of the quantity of the genetic material can be obtained.

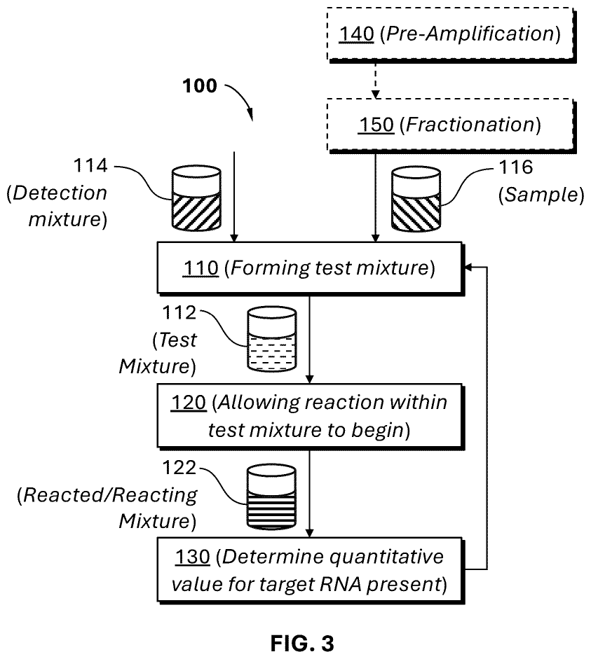

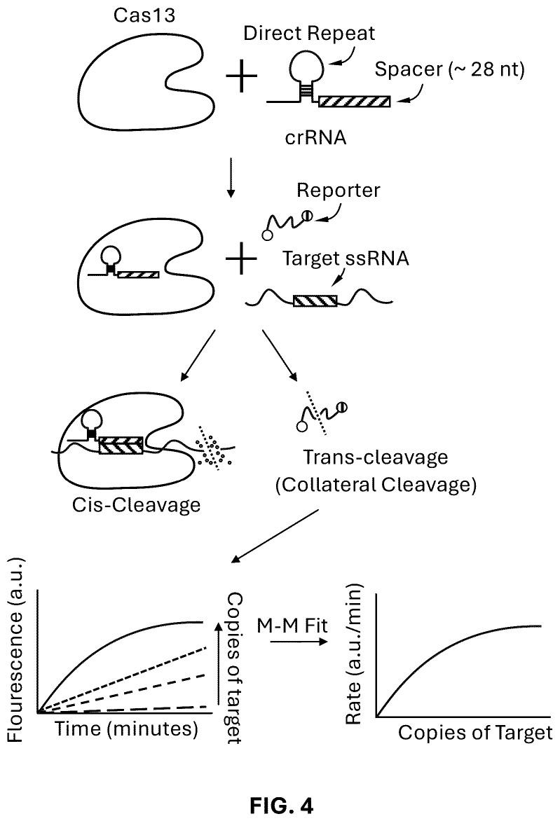

Referring to FIGS. 3 and 4, in various aspects, a method (100) for quantitative detection of a small, aberrant viral nucleic acid may be provided. Such small, aberrant viral nucleic acids may include synthetic RNA and/or RNA produced by a virus. It is envisioned that this may be used for the viral nucleic acids of any virus. Specific viruses may include, e.g., a coronavirus, an arenavirus, a hantavirus, a paramyxovirus, an Ebola virus, a picornavirus, a flavivirus, etc.

The method may include forming (110) a test mixture (112) by combining a detection mixture (114) with a sample (116). The sample may include an RNA sample.

The detection mixture may include a Cas13 enzyme, a reporter, a sequence-specific CRISPR RNA (crRNA), and a buffer. In a preferred embodiment, the detection mixture consists of those four components. In some embodiments, the detection mixture may comprise those four components.

The Cas13 enzyme may be one or more Cas13 enzymes. In some embodiments, it may be a single Cas13 enzyme. In some embodiments, it may be a plurality of Cas13 enzymes. The Cas13 enzyme may preferably be LbuCas13a or LwaCas13a. The Cas13 enzyme may be a Cas13 enzyme other than LbuCas13a or LwaCas13a. For example, the Cas13 enzyme may be Cas13a, LwaCas13a, LseCas13a, LbmCas13a, LbnCas13a, CamCas13a, CgaCas13a, Cga2Cas13a, Pprcas13a, LweCas13a, LneCas13a, Lwa2cas13a, RcsCas13a, RcrCas13a, RcdCas13a, LbuCas13a, RcaCas13a, EreCas13a, BzoCas13b, PinCas13b, PbuCas13b, AspCas13b, PsmCas13b, RanCas13b, PauCas13b, PsaCas13b, Pin2Cas13b, CcaCas13b, PguCas13b, PigCas13b, Pin3Cas13b and/or HheCas13a. The Cas13 enzyme may be present in an amount of, e.g., at least 1 nM, 2 nM, 3 nM, 4 nM, 5 nM, or 10 nM up to 10 nM, 20 nM, 50 nM, or 100 nM in the reaction mixture, including all appropriate subranges and combinations thereof.

The reporter may be any appropriate reporter. Such reporters may include, e.g., a fluorescent reporter (which may be, e.g., a quenched fluorescent reporter), a lateral flow reporter, a colorimetric reporter, and/or a luminescent reporter. Such reporters may include, e.g., an affinity-based reporter. Such reporters are well-known in the art. For example, fluorescein dyes (such as 5-Carboxyfluorescein (5-FAM) or 6-Carboxyfluorescein (6-FAM) may be coupled to a quencher molecule (such as a 3′ IOWA BLACK® FQ quencher molecule (3IABKFQ)) via a connector sequence of, e.g., 4-8 uricils. The reporter may be present in any appropriate amount, such as, e.g., at least 10 nM, 50 nM, 100 nM, 200 nM, or 250 nM up to 250 nM, 500 nM, 750 nM, or 1 μM in the reaction mixture, including all appropriate subranges and combinations thereof.

The sequence-specific crRNA may be configured to target a junction sequence formed upon aberrant viral RNA (such as mini viral RNA (mvRNA)) production. The crRNA may be present in an amount of, e.g., at least 1 nM, 2 nM, 3 nM, 4 nM, or 5 nM, up to 5 nM, 10 nM, 20 nM, 50 nM, or 100 nM in the reaction mixture, including all appropriate subranges and combinations thereof.

Any appropriate buffer may be used. For example, the buffer may include a known buffer, such as a phosphate buffer, a (4-(2-hydroxyethyl)-1-piperazineethanesulfonic acid) (HEPES) buffer, a tris(hydroxymethyl)aminomethane (TRIS)-hydrogen chloride (HCl) buffer, etc. Such buffers may be present in any appropriate amount, such as, e.g., at least 1 mM, 5 mM, 10 mM, 15 mM, or 20 mM up to 20 mM, 30 mM, 40 mM, 50 mM, or 100 mM in the reaction mixture, including all appropriate subranges and combinations thereof.

The buffer may include a charge carrier, such as sodium chloride (NaCl), potassium chloride (KCl), etc. Such charge carriers may be present in any appropriate amount, such as, e.g., 1 mM, 5 mM, 10 mM, 20 mM, 30 mM, 40 mM, 50 mM, or 60 mM up to 60 mM, 70 mM, 80 mM, 90 mM, 100 mM, or 250 mM in the reaction mixture, including all appropriate subranges and combinations thereof.

The buffer may include a precipitation and purification agent, such as polyethylene glycol (PEG). Such agents may be present in any appropriate amount, such as, e.g., at least 0.5%, 1%, 2%, 3%, 4%, or 5% up to 5%, 6%, 7%, 8%, 9%, or 10% of the reaction mixture, including all appropriate subranges and combinations thereof.

The buffer may include a co-factor agent, such as magnesium acetate (MgOAc). Such co-factor agents may be present in any appropriate amount, such as, e.g., 1 mM, 5 mM, or 10 mM up to 15 mM, 20 mM, 30 mM, 40 mM, or 50 mM in the reaction mixture, including all appropriate subranges and combinations thereof.

The detection mixture and/or reaction mixture may include RNAse inhibitor, such as RNAse inhibitor murine. RNAse inhibitor may be present in any appropriate amount, such as, e.g., at least 0.5 U/μL, 1 U/μL, 1.5 U/μL, or 2 U/μL up to 2 U/μL, 3 U/μL, 4 U/μL, 5 U/μL, or 10 U/μL in the reaction mixture, including all appropriate subranges and combinations thereof.

The method may include allowing (120) a reaction within the test mixture to begin. The method may include determining (130) a quantitative value (such as a fluorescence or lateral flow readout) representative of the reporter in the test mixture, where the quantitative value is a measure of an amount of target RNA present in the RNA sample.

The basic process is schematically illustrated in FIG. 4, Cas13 uses a CRISPR RNA (crRNA) that consists of a direct repeat region, which is specific to the Cas13 ortholog, and a spacer region, which is designed to be complementary to the target RNA. When CRISPR-Cas13 binds to the target RNA, the nuclease activity of Cas13 is activated. Cas13 can subsequently cleave the target, a process that is called cis cleavage, as well as any nearby single-stranded RNA molecules, a process called trans cleavage or collateral cleavage. The trans cleavage activity can be measured with a reporter RNA molecule (shown here as a quenched fluorescent reporter, containing a fluorophore and a quencher), making Cas13 suitable for a wide-range of applications, including the detection of genomic viral RNA. Additionally, the measured fluorescent signal is proportional to the amount of target RNA present in a sample and the number of target RNA molecules present in a reaction can be calculated using a standard curve specific for the target RNA.

### Example 1

In one example, reaction mixtures included 10 nM of either LbuCas13a or LwaCas13a, 20 mM HEPES pH 8.0, 60 mM KCl and 5% PEG, 2 U/μL RNAse inhibitor murine (New England Biolabs), 0.25 μM 6UFAM (From 5′ to 3′:/6-FAM/UUUUUU (SEQ ID NO. 1)/3IABKFQ/), 14 mM MgOAc, 5 nM crRNA (see Table 1), and the reported amount of target RNA. Each reaction was first combined in a volume of 44 μL in 96-well plates. After mixing, 20 μL was transferred in duplicate to 384-well plates. The plate was then placed in a BioTek Cytation 5 Cell Imaging Multi-Mode Reader (Agilent) or a BioTek Synergy H1 Plate Reader and incubated at 37° C. for 3 hours. Fluorescence was measured every 5 minutes.

TABLE 1

| | | | Limit of Detection (copies) |
|---|---|---|---|
| | | | (lowest standard that was 3 |
| | | Sequence | standard deviations above |
| Name | Target | (5′ to 3′) | mock) |
| CL06 | Human | GAUUUAGACUACCCCAAAAAC | Not determined |
| | 5s rRNA | GAAGGGGACUAAAACGGGCG | |
| | | CGUUCAGGGUGGUAUGGCCG | |
| | | UAG (SEQ ID NO. 2) | |
| CL11 | Human | GACCACCCCAAAAAUGAAGGG | Not determined |
| | 5s rRNA | GACUAAAACGGGCGCGUUCA | |
| | | GGGUGGUAUGGCCGUAG (SEQ | |
| | | ID NO. 3) | |
| CL09 | NP71.6 | GACCACCCCAAAAAUGAAGGG | 3e5 |
| | | GACUAAAACUAGACUAGUGG | |
| | | CAACCAAAACGGCCGGA (SEQ | |
| | | ID NO. 4) | |
| CL63/A | NP-61 | GACCACCCCAAAAAUGAAGGG | 8e6 |
| | | GACUAAAACATCACTCACAGA | |
| | | GTGACATCGAAAAATA (SEQ | |
| | | ID NO. 5) | |
| CL51/B | NP-61 | GACCACCCCAAAAAUGAAGGG | 3e5 |
| | | GACUAAAACAGAUAAUCACU | |
| | | CACAGAGUGACAUCGAA (SEQ | |
| | | ID NO. 6) | |
| CL66/C | NP-61 | GACCACCCCAAAAAUGAAGGG | 4e7 |
| | | GACUAAAACACATCGAAAAAT | |
| | | ACCCTTGT (SEQ ID NO. 7) | |
| CL67/D | NP-61 | GACCACCCCAAAAAUGAAGGG | 4e7 |
| | | GACUAAAACACAGAGTGACAT | |
| | | CGAAAAAT (SEQ ID NO. 8) | |
| CL60/E | NP-61 | GACCACCCCAAAAAUGAAGGG | 1e6 |
| | | GACUAAAACAGAGTGACATCG | |
| | | AAAAATACCCTTGTTT (SEQ ID | |
| | | NO. 9) | |
| CL61/F | NP-61 | GACCACCCCAAAAAUGAAGGG | 1e6 |
| | | GACUAAAACACAGAGTGACAT | |
| | | CGAAAAATACCCTTGT (SEQ ID | |
| | | NO. 10) | |
| CL62/G | NP-61 | GACCACCCCAAAAAUGAAGGG | 8e6 |
| | | GACUAAAACAGTGACATCGAA | |
| | | AAATACCCTTGTTTCT (SEQ ID | |
| | | NO. 11) | |
| CL65/H | NP-61 | GACCACCCCAAAAAUGAAGGG | 8e6 |
| | | GACUAAAACCAGAGTGACATC | |
| | | GAAAAATA (SEQ ID NO. 12) | |
| CL64/I | NP-61 | GACCACCCCAAAAAUGAAGGG | 8e6 |
| | | GACUAAAACTGACATCGAAAA | |
| | | ATACCCTT (SEQ ID NO. 13) | |
| CL68 | NP71.11 | GACCACCCCAAAAAUGAAGGG | 8e6 |
| | | GACUAAAACTAGTTACCCTGC | |
| | | TTTTGCTGCCACTAGT (SEQ ID | |
| | | NO. 14) | |
| CL69 | NP71.11 | GACCACCCCAAAAAUGAAGGG | 8e6 |
| | | GACUAAAACTAGTATGGGTGC | |
| | | TTTTGCTGCCACTAGT (SEQ ID | |
| | | NO. 15) | |

Referring again to FIG. 3, the method may be free of an amplification step. In some aspects, the method may optionally include pre-amplification (140) of the RNA sample. Such pre-amplification steps are well understood in the art; any appropriate pre-amplification technique may be utilized here, based on the sample involved.

The method may optionally include RNA fractionation (150). RNA fractionation techniques are well understood in the art; any appropriate RNA fractionation technique may be utilized here, based on the sample involved.

In some aspects, the sample may include an antiviral (e.g., an antiviral known or expected to inhibit the virus), and the method may include monitoring an infection status by repeating at least the forming, allowing, and determining steps and tracking the quantitative value over time.

In various aspects, a composition of matter (e.g., detection mixture (114) or test mixture (112)) may be provided. As disclosed herein, the composition of matter may include a Cas13 enzyme, a reporter, a sequence-specific CRISPR RNA (crRNA), and a buffer. In some aspects, the composition of matter may include sample RNA.

In various aspects, a detection system may be provided. The detection system may include a detection mixture container, where a composition of matter as disclosed herein may be disposed within the detection mixture container. The container may be any appropriate container for holding the detection mixture and optionally the reaction mixture. This may include, e.g., a flask, beaker, jar, etc., and may include, e.g., one or more wells in an array of wells. The detection system may include a detector configured to detect fluorescence of the composition of matter. For example, the detection system may include a plate reader.

In various aspects, a detection kit may be provided. The detection kit may include a sample container and a detection mixture container. The container may be any appropriate container for holding the detection mixture and optionally the reaction mixture. This may include, e.g., a flask, beaker, jar, etc. A composition of matter as disclosed herein may be disposed within the detection mixture container.

### Example—Influenza a Virus (IAV)

During IAV infection, aberrant or nonstandard RNA molecules of various lengths and sequences are produced. A subset of these molecules can activate the innate immune response, making them important targets for in-depth characterization. It has been observed that routinely used RNA quantification methods are sensitive to the viral RNA sequence and that they can fail to correctly measure viral RNA levels and potentially create additional aberrant signals. The CRISPR-associated enzyme Cas13 is explored herein as an additional tool to quantify aberrant viral RNA levels in cell culture, animal model, and patient samples. Specifically, this example focuses on the detection and quantification of mvRNAs, which are sufficiently short to synthesize chemically and thus benchmark properly.

Amplification of mvRNAs by RT and PCR Introduces Errors.

Different detection methods can be used to quantify IAV aberrant RNA species, but their errors have not previously been compared directly for myRNAs. To gain insight into the errors generated by these methods, the ability of RT and PCR to quantify known, chemically synthesized mvRNA sequences can be compared. Using two mvRNAs of the same length, but with different internal sequences, a lack of consistency was observed in the ability of three different RT and PCR enzyme combinations to produce a single amplification product. Additionally, the different RT and PCR enzyme combinations amplified the mvRNAs to different levels even though a fixed amount of the synthetic mvRNAs was provided as input. Thus, while primer extension, PCR, and next generation sequencing are powerful methods to visualize or discover aberrant IAV RNAs, their use as quantification methods appears limited by the enzymes used for the conversion or amplification of different mvRNA sequences.

LbuCas13a can be Used to Detect and Quantify mvRNAs without Amplification.

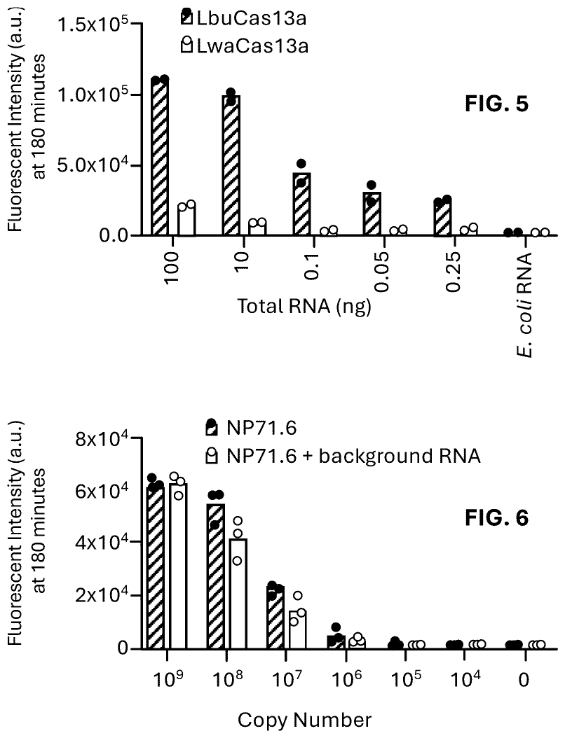

Cas13 can be used to detect SARS-COV-2 RNA without (isothermal) amplification and T7 transcription steps when the genome is targeted by multiple guides. As only one unique junction is available in mvRNA sequences (see FIGS. 2, 4) the previously described approach is not feasible for IAV aberrant RNA molecules. However, Cas13 orthologs have variable amounts of trans-cleavage activity and this activity can be used to generate additional signal. To determine if Cas13 could potentially be used to detect mvRNA junctions without requiring amplification or T7 transcription steps, the ability of two Cas13 orthologs, LbuCas13a and LwaCas13a, to directly detect human 5S rRNA in a dilution series of total RNA extracted from HEK293T cells was first compared. It was observed that LbuCas13a was able to detect 5S rRNA with as little as 0.025 ng of total RNA input, whereas LwaCas13 only produced a signal above background when incubated with 10 ng of total RNA input. See FIG. 5.

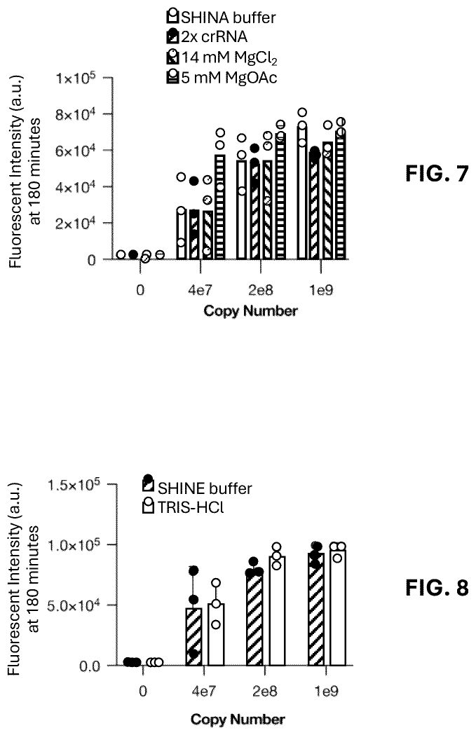

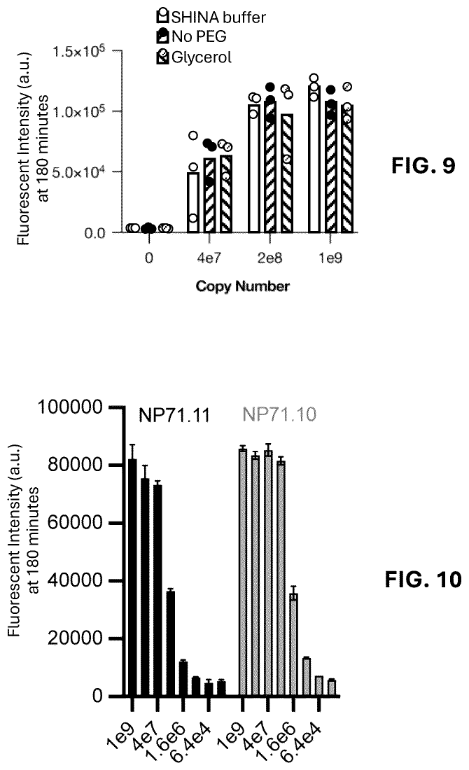

It was next investigated whether a Cas13 enzyme, such as LbuCas13a, could detect a previously described engineered mvRNA based on segment 5 (NP71.6). To detect NP71.6, a crRNA (CL09) was designed with a spacer sequence complementary to the unique junction sequence relative to the full-length segment 5 vRNA. Chemically synthesized NP71.6 was subsequently diluted in water or 100 ng of total HEK293T RNA and it was found that the mvRNA was able to be detected with a limit of detection <10⁶ copies, irrespective of the background used. See FIG. 6. No fluorescent signal was observed in the absence of synthetic NP71.6. See FIG. 6. For the reactions, the known LwaCas13a SHINE buffer (see, e.g., Example 1) was used, and it was confirmed that the crRNA, Mg²⁺, PEG, and glycerol conditions of the SHINE buffer were also optimal for LbuCas13a reactions. See FIGS. 7-9. Any buffer component substitutions that was explored did not yield significant changes. Using the same approach, it was also found that Cas13 can detect the mvRNAs and NP71.10 and NP71.11 (see Table 2, below). See FIG. 10.

TABLE 2
Example mvRNAs

| | Sequence |
|---|---|
| Name | (5′ to 3′) |
| NP71.10 | AGUAGAAACAAGGGUAUUUUUCUUUACUAGUGGCAGCAAA |
| | AGCAGGGUAACUAGUCUACCCUGCUUUUGCU (SEQ ID NO. |
| | 16) |
| NP71.11 | AGUAGAAACAAGGGUAUUUUUCUUUACUAGUGGCAGCAAA |
| | AGCACCCAUACUAGUCUACCCUGCUUUUGCU (SEQ ID NO. |
| | 17) |
| NP71.6 | AGUAGAAACAAGGGUAUUUUUCUUUACUAGUCCGGCCGUU |
| | UUGGUUGCCACUAGUCUACCCUGCUUUUGCU (SEQ ID NO. |
| | 18) |
| NP61 | AGUAGAAACAAGGGUAUUUUUCGAUGUCACUCUGUGAGUG |
| | AUUAUCUACCCUGCUUUUGCU (SEQ ID NO. 19) |
| mvRNA75 | AGTAGAAACAAGGGCACTACTGGAAAACTACCTGTTCCATGG |
| | CCAACACTTGTCACTACTTTCCCTGCTTTTGCT (SEQ ID NO. |
| | 20) |
| mvRNA100 | AGTAGAAACAAGGGTAACAGCTGCTGGGATTACACATGGCA |
| | TGGATGAACTATACAAATAAATGTCCAGACCTGCAGGCATGC |
| | AAGCTCCTGCTTTTGCT (SEQ ID NO. 21) |
| mvRNA120 | AGTAGAAACAAGGGCGATGGCCCTGTCCTTTTACCAGACAAC |
| | CATTACCTGTCCACACAATCTGCCCTTTCGAAAGATCCCAAC |
| | GAAAAGAGAGACCACATGGTCCTTCCTGCTTTTGCT (SEQ ID |
| | NO. 22) |

LbuCas13a can be Used to Quantify mvRNAs in Total Cell RNA.

It was subsequently explored whether one could use a Cas13 enzyme, such as LbuCas13a, to quantify unknown amounts of NP71.6 in total RNA extracted from HEK293T cells expressing the IAV mini-genome system (Te Velthuis et al. 2018b). In this assay, plasmids expressing the three wildtype (WT) IAV RNA polymerase subunits via an RNA polymerase II (Pol II) promoter and the NP71.6 mvRNA via Pol I promoter were transfected into HEK293T cells.

For plasmids used in all of these examples, the pcDNA3-based WSN protein expression plasmids and pPolI-based WSN template RNA expression plasmids were described previously (Te Velthuis et al. 2018a; French et al. 2022). The mvRNA expressed plasmids were generated by site-directed mutagenesis using the primers listed in Table 2.

For all of these examples, to prepare synthetic RNA and primers, primers were synthesized by Integrated DNA Technologies (IDT) and resuspended in nuclease-free water to 100 μM. Primers were stored at −20° C. and were further diluted prior to analysis. crRNAs were synthesized by IDT and resuspended in nuclease-free water to 100 μM. crRNAs were stored at −70° C. and were further diluted prior to analysis. Synthetic RNA targets were synthesized by IDT and resuspended in nuclease-free water to 100 μM. Synthetic RNA targets were stored at −70° C. and were further diluted prior to analysis.

TABLE 3
Primers and other oligonucleotides

| | | Sequence |
|---|---|---|
| Name | Description | (5′ to 3′) |
| GC- | Targets NP | AGCAAAAGCAGGGTAGACTAGT (SEQ ID NO. 23) |
| | segment | |
| NP 5′ | Targets NP | AGTAGAAACAAGGGTATTTTTC (SEQ ID NO. 24) |
| | segment | |
| GC50.9 | Targets NP71.6 | /FAM/TTACTAGTC (SEQ ID NO. 25)/ZEN/ |
| probe | | CGGCCGTTTTGGTTGC (SEQ ID NO. 26)/3IABKFQ/ |
| Lv3ga | VRNA RT primer | GTTCAGACGTGTGCTCTTCCGATCTAGCG (SEQ ID |
| | (contains LNA) | NO. 27) + AAAGCAGG (SEQ ID NO. 28) |
| Lv3aa | vRNA RT primer | GTTCAGACGTGTGCTCTTCCGATCTAGC (SEQ ID NO. |
| | (contains LNA) | 29) + A + AAAGCAGG (SEQ ID NO. 30) |
| Lv5 | vRNA forward | CACGACGCTCTTCCGATCTHNNNNNNNAGTAGAA |
| | primer (contains | (SEQ ID NO. 31) + A + CAAGG (SEQ ID NO. 32) |
| | LNA) | |
| P5short | vDNA fw | GAGATCTACACTCTTTCCCTACACGACGCTCTTCCG |
| | | ATCT (SEQ ID NO. 33) |
| I7short | vDNA rv | GAGATACTGGTGTGACTGGAGTTCAGACGTGTGCTC |
| | | TTCCGATCT (SEQ ID NO. 34) |

For all of these examples, transfections of HEK293T cells were performed using Lipofectamine 2000 (Invitrogen) and Opti-MEM (Invitrogen) as described previously (Te Velthuis et al. 2018a; French et al. 2022).

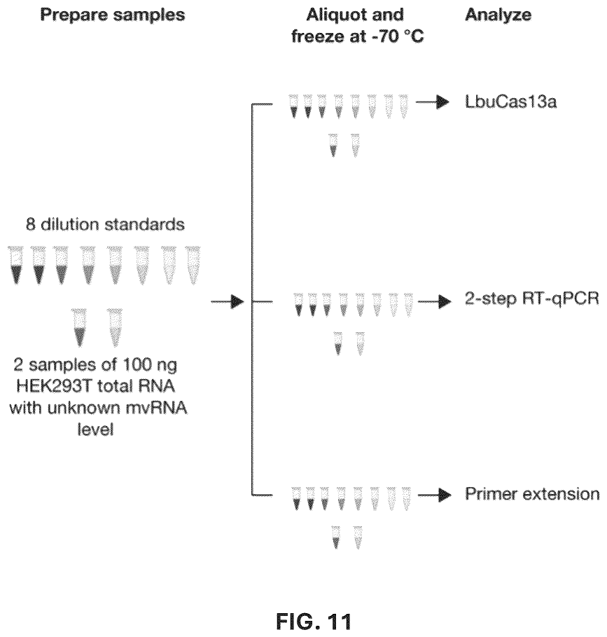

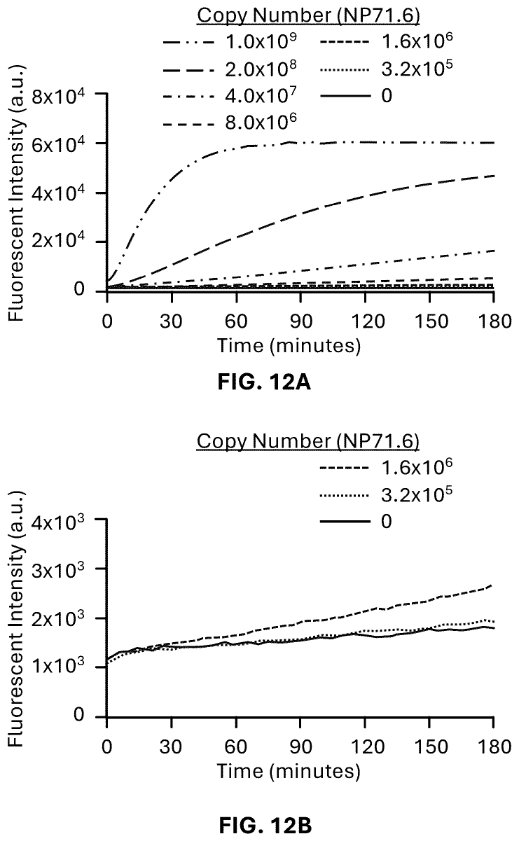

As control, a PB1 RNA polymerase subunit that contained alanine substitutions at position D445 and D446 (PB1a) instead of the WT PB1 subunit was transfected. Total RNA was extracted 48-hours post transfection. To investigate the ability to determine the mvRNA level in HEK293T cells, a 5-fold dilution series of chemically synthesized NP71.6 in total RNA was first made from untransfected HEK293T cells. This dilution series was the standard curve. See FIG. 11, 12A-12B.

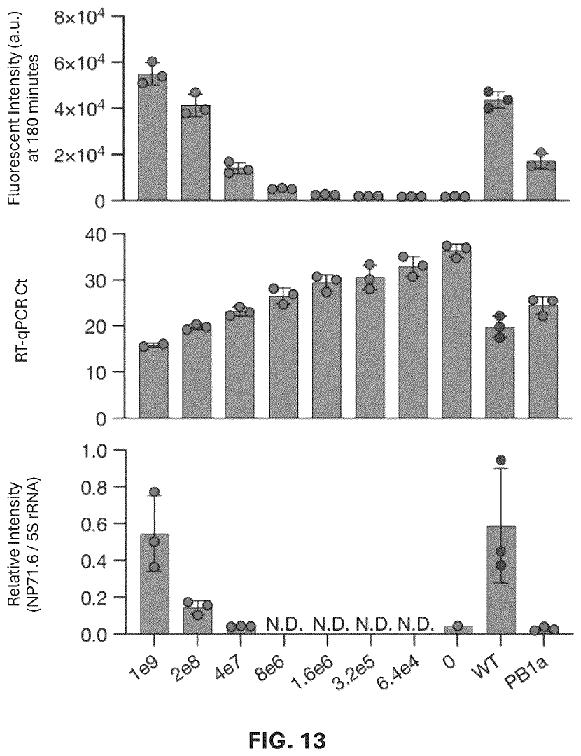

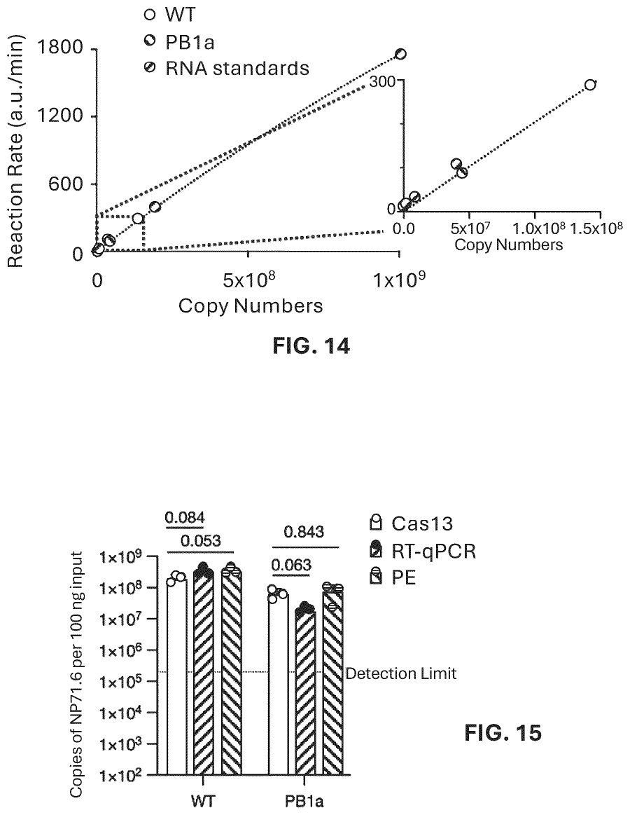

Next, the Cas13 fluorescent signal was measured (see FIG. 13), the maximum slope of the fluorescent Cas13 signal for each point of the standard curve was calculated, the maximum slope was plotted against the known synthetic mvRNA concentrations, and the resulting distribution was fitted to the Michaelis-Menten equation. See FIG. 14.

Specifically, for curve fitting and quantification in these examples, the maximum slope of each sample and standard was obtained by first determining the slope of every 3 data points starting at t=0 min (0-10 min, 5-15 min . . . 170-180 min). The maximum slopes of the standards were then plotted against the known copy numbers of the standards. Standards that reached saturation or quickly reached saturation (slopehigh-concentration <<slopelow-concentration) were omitted prior to fitting the curve. The curve was fit to the Michaelis-Menten equation and Kₘ and Vₘₐₓ values were estimated using the Python 3 and the curve-fit trust region reflective (TRF) method from the scipy.optimize package. The Vₘₐₓ and Kₘ values were also obtained using Prism 10 software, both Python and Prism 10 software produced the same Vₘₐₓ and Kₘ values. Using the obtained Kₘ and Vₘₐₓ values and the maximum velocity (V), referred to as the reaction rate in the text, of the sample, the unknown concentrations of the samples were estimated. Limits of detection for each crRNA are listed in Table 1.

Next, the Cas13 signal of the transfected HEK293T RNA was determined and the fitted standard curve data used to convert the fluorescent signal of the transfected HEK293T cells into mvRNA copy numbers. See FIGS. 13, 15.

It was shown that RT-PCR detection methods can introduce mvRNA amplification biases. To reduce this bias and detect mvRNAs using RT-qPCR in a sequence-specific manner, similar to the Cas13 assay, a TaqMan probe was introduced that was specific for the junction in the NP71.6 mvRNA. When the quantification obtained using LbuCas13a was next compared to the results obtained using primer extension or a two-step TaqMan-based RT-qPCR (see FIG. 13). It was observed indeed that the disclosed CRISPR-based detection method yielded no significantly different results. See FIG. 15. A similar result was obtained when the NP71.6 mvRNA was expressed in the presence of an inactive IAV RNA polymerase to measure RNA polymerase I (PolI)-derived input levels. See FIG. 15. Consequently, for a single, well-defined IAV mvRNA the RT-qPCR and Cas13 assays yield comparable results. However, in infection experiments, the mvRNA diversity is relatively large, making it more likely that sequence-based biases arise when amplification steps are used for myRNA detection.

Trade-Off Between LbuCas13a crRNA Sensitivity and Specificity.

It was next explored whether LbuCas13a could also detect an authentic, conserved, and broadly expressed mvRNA. In addition, it was desired to explore how different crRNAs affect LbuCas13a's ability to detect an mvRNA, and how well a single crRNA can distinguish this NP-61 from other viral sequences, such as the full-length genome segment. For this example, the inventors focused on a 61 nt-long segment 5 mvRNA (NP-61). This mvRNA is highly abundant and present in ferret lung tissue infected with A/Brevig Mission/1/1918 (H1N1) or A/Indonesia/2005 (H5N1) or A549 cells infected with A/WSN/1933 (H1N1) (abbreviated as WSN).

To address the above points, 9 different crRNAs were designed to detect NP-61 with spacer lengths that varied from 20 to 28 nt. (see Table 1, A-H, starting at CL63/A. Next, the ability of each crRNA was tested to detect chemically synthesized NP-61, a T7 transcript of full-length segment 5, and viral RNA in IAV infected cells after fractionation into >200 and <200 nt RNA (thereby focusing detection on full-length and DVGs or mvRNAs, respectively).

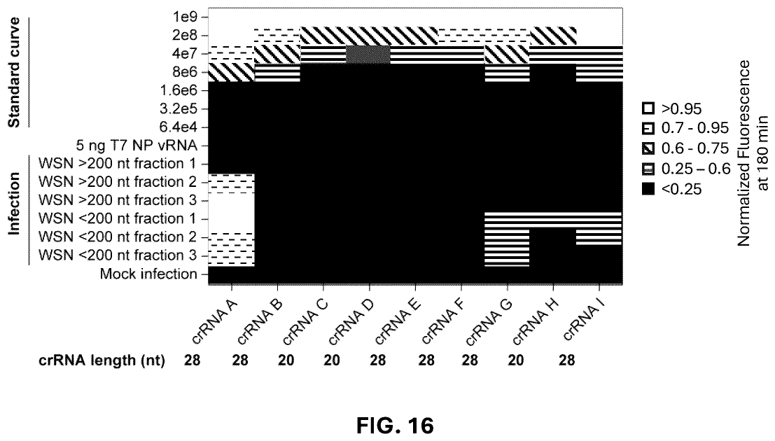

As shown in FIG. 16, it was found that the nine crRNAs detected the synthetic NP-61 with different sensitivities and thus limits of detection (LOD), which was determined from the lowest concentration of the standard curve that can be detected 3-standard deviations above the negative control (see Table 1). The three most sensitive guides were crRNAs A, B and G (FIG. 16, Table 1).

To test which of the crRNAs cross-reacted with full-length segment 5 vRNA, in vitro transcription of the segment 5 vRNA was performed. Using primer extension, it was determined that ˜5 ng of transcript was representative of the segment 5 vRNA level present in a WSN infection. When the crRNAs were subsequently tested against 5 ng transcript in the presence of 50 ng A549 background RNA, it was found that none of the crRNAs generated a significant signal (see FIG. 16). It was therefore concluded that there is none or only limited cross-reactivity with the full-length segment 5 vRNA.

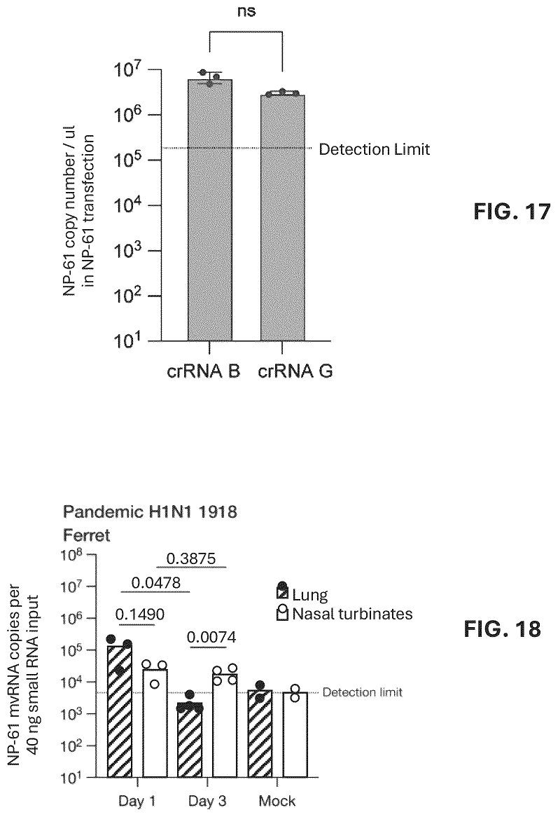

Analysis of fractionated infection samples showed that only guides A and B yielded high signals for the <200 and >200 nt fractions. Since it was expected the large fraction only contain full-length and DVG RNA, and the crRNAs did not support detection of full-length segment 5 vRNA, it is suspected that cross-reactivity against segment 5 DVG species had occurred. crRNA G was the only cRNA that yielded a consistent signal above the LOD in all three <200 nt fractions. The signal produced with crRNA G for the <200 nt RNA fraction was lower than the signals obtained with crRNAs A and B. Since the LOD of crRNAs A and B was not substantially different from crRNA G, this result suggests that crRNAs A and B likely trigger cross-reactivity with other mvRNAs derived from segment 5. However, as mvRNAs are diverse in sequence and it is not reasonably feasible to chemically synthesize all sequence permutations, one cannot exhaustively test its specificity and must thus assume that crRNA G had limited cross-reactivity with other mvRNAs. To at least confirm that a single authentic mvRNA species can be detected with the same efficiency by cRNAs B and G in a defined context, NP-61 was transfected into HEK 293T cells and total RNA was extracted. As shown in FIG. 17, quantification of the NP-61 levels in the transfection sample yielded similar results with the two crRNAs.

Overall, these results suggest that there is likely a trade-off, as in any other nucleic acid detection assay, between crRNA specificity and sensitivity when detecting mvRNAs in complex samples. Since it is impossible to test each guide against all possible segment 5 mvRNAs sequences, we conclude that the detection of a single mvRNA species in a complex sample is likely not possible. In the downstream experiments, we therefore focused on the detection segment 5 mvRNAs with the best sensitivity possible after fractionation to remove any DVG sequences.

LbuCas13a can Detect mvRNAs in RNA Extracted from Ferret Lungs.

It has previously been shown that mvRNAs can be detected in mouse and ferret lung samples using RT-PCR and next generation sequencing. Thus, it was next sought to determine whether Cas13 could be used to detect mvRNAs in RNA extracted from ferret lung or nasal turbinate tissues infected with pandemic A/Brevig Mission/1/1918 (H1N1).

Based on the results shown in FIGS. 16-17, it was suspected that crRNA G would not be sensitive enough to detect mvRNAs. Therefore, the RNA was fractionated and Cas13 detection was performed using crRNA B. As shown in FIG. 18, crRNA B was able to detect mvRNA in the <200 nt sample in ferret lung or nasal turbinate tissues on day 1, and in the nasal turbinates on day 3 post infection. Variation in myRNA abundance between the infected ferrets was observed. A significant difference in mvRNA abundance was observed between the lung and nasal turbinate homogenates on day 3.

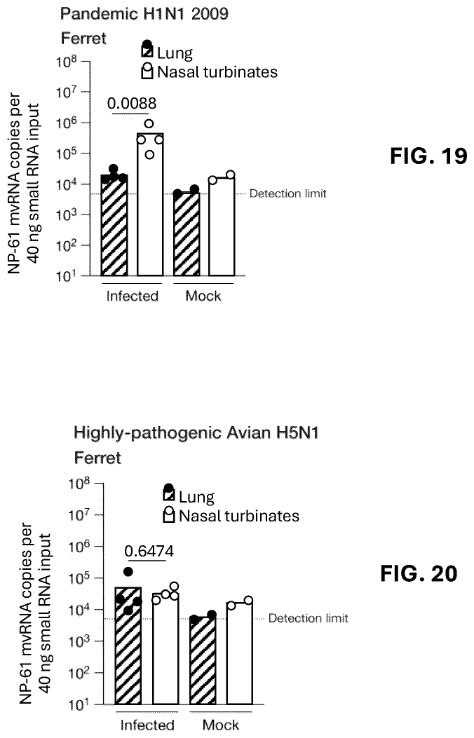

mvRNA sequences vary among IAV strains as the sequences downstream of the promoter are not fully conserved. However, based on previous deep-sequencing data (French et al. 2022), it was found that NP-61 was also produced by the pandemic A/Netherlands/602/2009 (H1N1) and highly pathogenic avian A/Indonesia/5/2005 (H5N1) isolates. The relative abundance of the NP-61 mvRNA and cross-reacting species in ferret lung or nasal turbinate homogenates infected with these two strains was therefore measured. As shown in FIGS. 19 and 20, the disclosed technique was able to detect mvRNAs in the infected respiratory tract tissues infected by the two IAV strains. A significant difference in mvRNA abundance between the lung or nasal turbinate homogenates was observed in the pandemic H1N1 2009 infections.

LbuCas13a can Detect mvRNAs in RNA Extracted from Clinical Swabs.

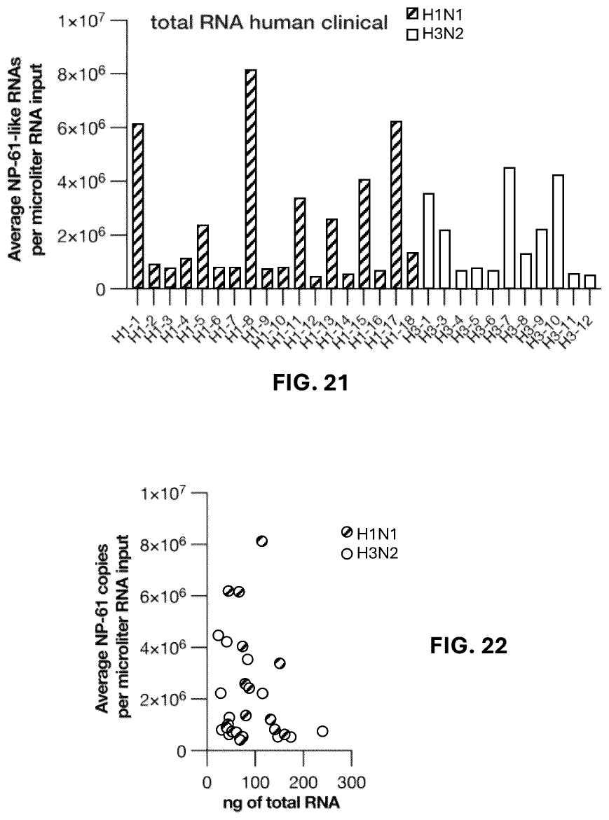

Finally, it was investigated whether the disclosed technique could detect mvRNAs in RNA extracted from nasopharyngeal swabs from patients who had been confirmed to infected with seasonal IAV. The clinical samples obtained were either positive for seasonal H1N1 or H3N2, and were not co-infected with other respiratory pathogens, as determined by clinical RT-qPCR. It was first confirmed that the disclosed method could detect mvRNAs in clinical samples using a RT-PCR and found signals corresponding to the expected size in 6 of 10 IAV positive clinical samples. Next, crRNA B was used to detect segment 5 RNAs (DVG and mvRNAs) in unfractionated RNA as well as mvRNAs in fractionated RNA. Positive signals above the limit of detection were detected for 29 of the 30 clinical samples tested when using unfractionated RNA. This suggests that without fractionation, crRNA B could be used for amplification-free detection of influenza A virus infection. Using crRNA B and a synthetic NP-61 standard curve, the signals were converted to number of NP-61-like viral RNA copies (i.e., mvRNA and DVG) per μl of extracted clinical sample. This type of copy number was not correlated with the clinical RT-qPCR Ct value. See FIGS. 21 and 22.

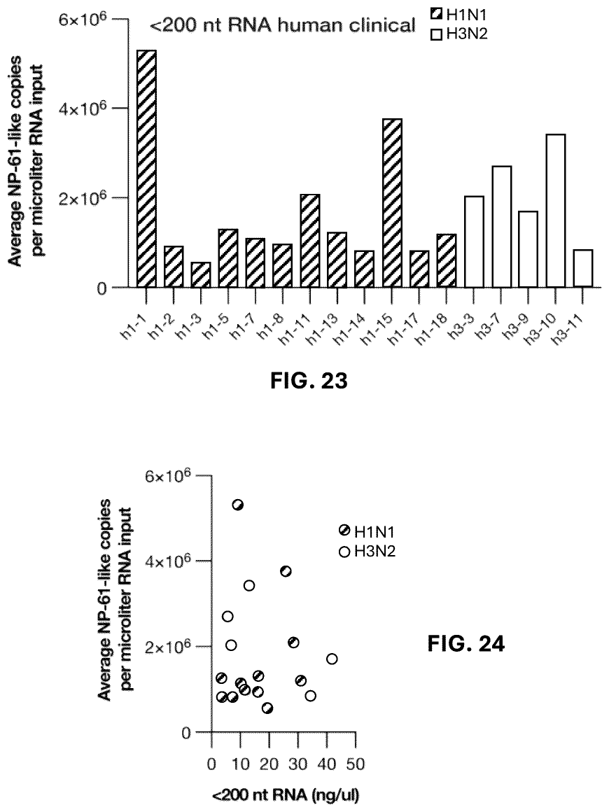

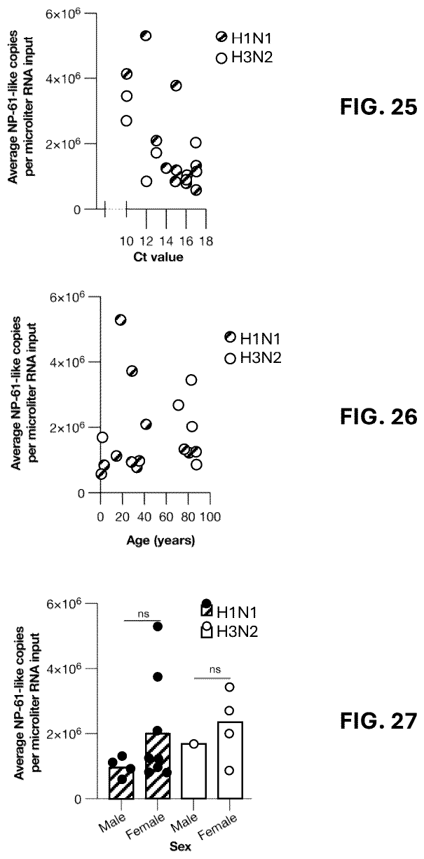

To get an estimate of the NP-61-like mvRNA copies per μl of extracted clinical sample, Cas13-based detection was performed on the fractionated samples. Positive signals were observed in fractionated samples for 16 of 17 clinical samples tested. Using the synthetic NP-61 standard curve, the number of NP-61-like mvRNA copies per μl of extracted clinical sample were calculated. See FIG. 23. Further analysis showed that these copy numbers were not correlated with the amount of RNA input (see FIG. 24), the reported clinical RT-qPCR Ct value (see FIG. 25), or patient age (see FIG. 26). No significant difference was observed among the H1N1 samples with respect to the reported patient's sex (see FIG. 27). Overall, these findings show that mvRNAs can be detected in a wide range of samples, including, e.g., influenza virus positive clinical samples, but quantification of a single mvRNA sequence requires input quantities of RNA that cannot be achieved using animal model or clinical samples.

In line with previous reports, it was shown that the LbuCas13a ortholog can detect and quantify viral RNAs without amplification in defined samples that contain a relatively high amount of RNA. Only a single crRNA was used, because for this example, it was specifically desired to target the unique junctions in an mvRNA. It was found that without using multiple crRNAs, the Cas13-based approach can detect a single mvRNA sequence or several closely related sequences if one opts to use a crRNA that has a higher sensitivity. A limitation of the Cas13 approach is the sensitivity to point mutations, in particular when using sensitive crRNAs. This may be an issue for rapidly evolving RNA viruses, which can quickly accumulate point mutations. This issue may be overcome by using occluded Cas13, which is a new approach that makes Cas13 more sensitive to single nucleotide changes.

For a sample containing a single mvRNA expressed in cells and amplified by the IAVRNA polymerase, LbuCas13a quantifications yielded results that were not significantly different from the primer extension assay. This result was expected, as any additional aberrant products produced by the IAV RNA polymerase in cell culture were excluded in the PAGE-based size selection that underlies the primer extension analysis. Similarly, the TaqMan-based RT-qPCR produced results that were not significantly different from the Cas13-based assay, because the TaqMan probe hybridized to the mvRNA junction similar to the crRNA of Cas13 and thus also excluded any aberrant PCR product that did not include the junction. For samples where RNA levels are low, a TaqMan-based RT-qPCR assay may be preferable as the LbuCas13 approach currently has a relatively high detection limit. Different combinations of enzymes can be tested to find the most accurate amplification-based assay. These observations are likely also important for studies of DVG sequences, which also rely on RT-PCR-based sequencing libraries to study sequence variations or identify DVG junctions.

Using LbuCas13a, the copy number of a single mvRNA in transfected cells were quantified (see FIGS. 14-15) and WSN-infected cells (see FIGS. 16-17). In infection samples with low RNA concentrations, only a group of mvRNAs that was related in sequence to a highly abundant mvRNA could be detected, because the most sensitive crRNAs have cross reactivity with other aberrant viral RNAs. Although this finding is disappointing and defines a clear limitation for the assay, one is able to detect an mvRNA signal in fractionated clinical samples that were positive for H1N1 and H3N2 seasonal IAV RNA. This result demonstrates that mvRNAs are present in human samples for the first time.

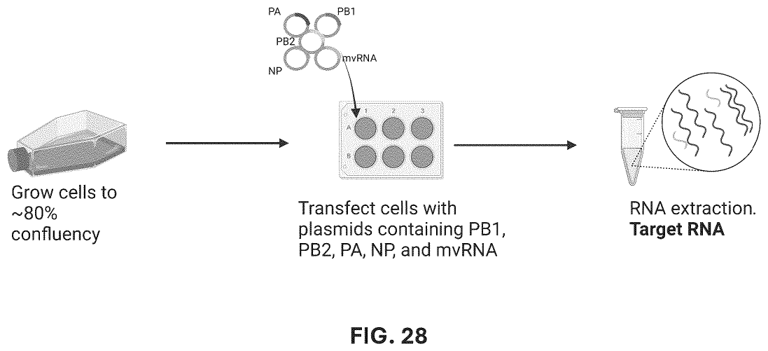

FIG. 28 schematically illustrates an mvRNA transfection protocol, including growing cells to around 80% confluency, transfecting cells with plasmids containing PB1, PB2, PA, NP, and mvRNA, followed by RNA extraction (including the Target RNA).

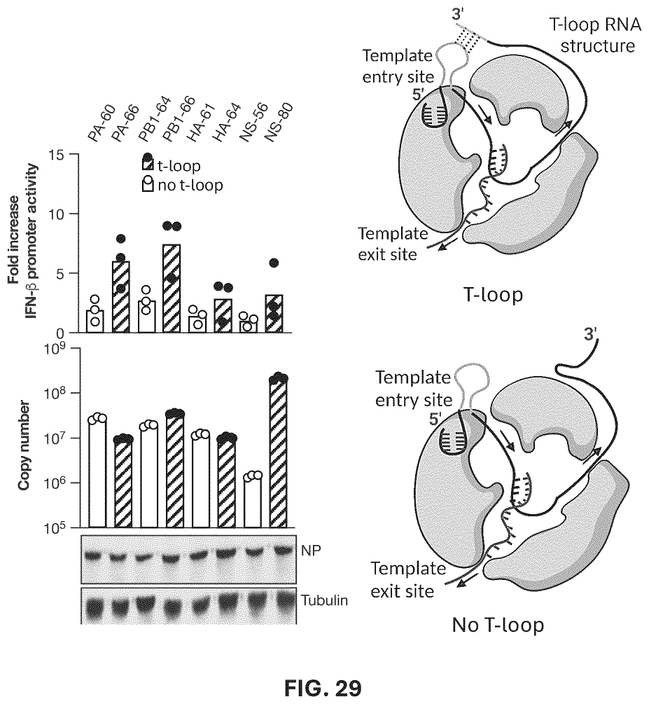

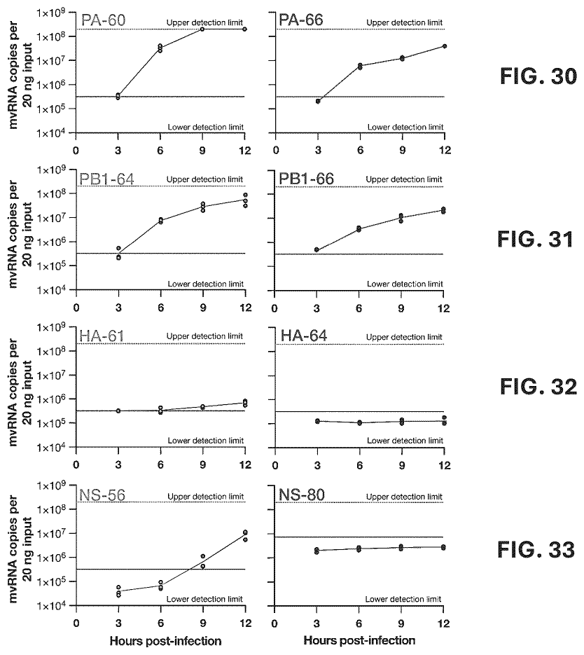

FIG. 29 shows the effect of different mvRNA sequences on the IFN response. As seen, the t-looped sequences generally have higher fold increases in IFN-β promoter activity over comparable non t-looped sequences (e.g., PA-60 vs PA-66, PB1-64 vs PB1-66, HA-61 vs HA-64, and NS-56 vs NS-80). The reaction kinetics can be seen in FIGS. 30-33, showing, in the hours post-infection, the differences in mvRNA copies per 20 ng input for the various sequences.

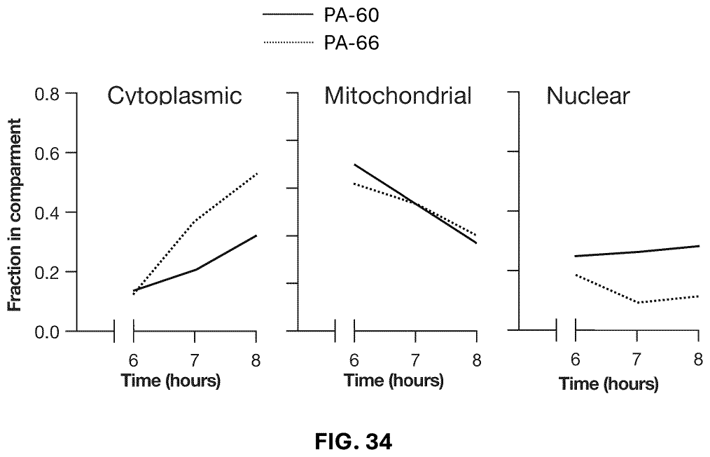

The disclosed method can also be used to determine localization of mvRNA sequences within different compartments of a cell. For example, referring to FIG. 34, the fraction of PA-60 vs PA-66 within cytoplasmic, mitochondrial, and nuclear compartments can be tracked, the disclosed approach in combination with traditional techniques, e.g., for isolation and RNA extraction.

Further, it is recognized that certain treatments, including antimetabolites such as fluorouracil, may induce mvRNA formation. Mutations that may resist some treatments, such as a ribavirin (nucleoside analog) resistance mutation (PB1 V43I) may inhibit or otherwise lead to less mvRNA generation.

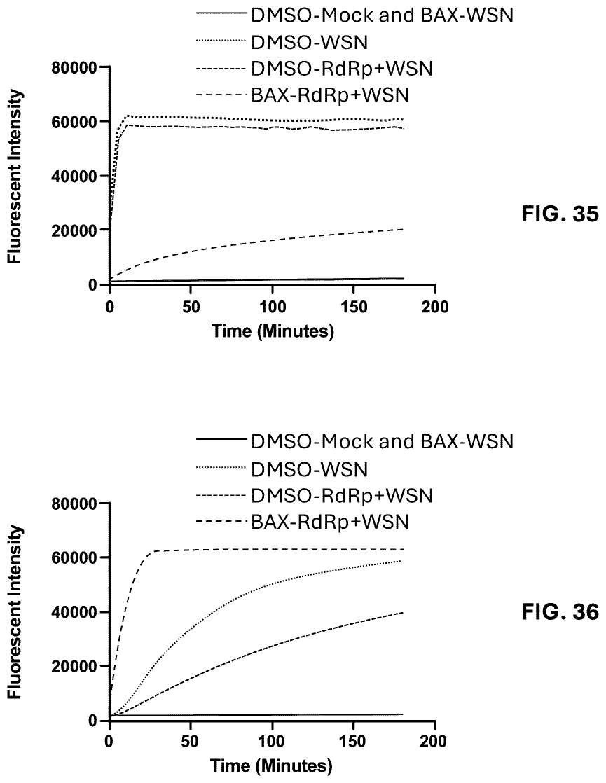

Treatments such as beloxavir marboxil (baloxavir, or BAX) are used to treat and prevent influenza. Baloxavir inhibits IAV transcription, and as a result the production of RdRp. Baloxavir treatment and adding RdRp allows for replication while inhibiting transcription. This can be seen with reference to FIGS. 35 and 36, where the fluorescent intensities of various combinations of DMSO or BAX with WSN (A/WSN/1933 (H1N1)), Mock, or RdRp and WSN we considered for 180 minutes, for V-sense (FIG. 35) and C-sense (FIG. 36) strands.

The direct detection of aberrant RNA can be readily achieved for other viruses as well. For example, Hantavirus involves a negative-sense RNA virus with segmented genome (three segments, a small(S), medium (M), and large (L)), where transmission can occur from small mammals to humans.

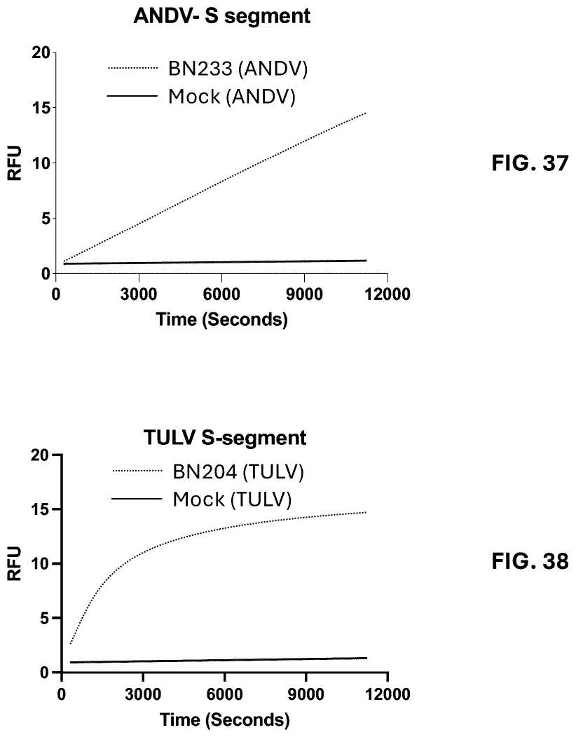

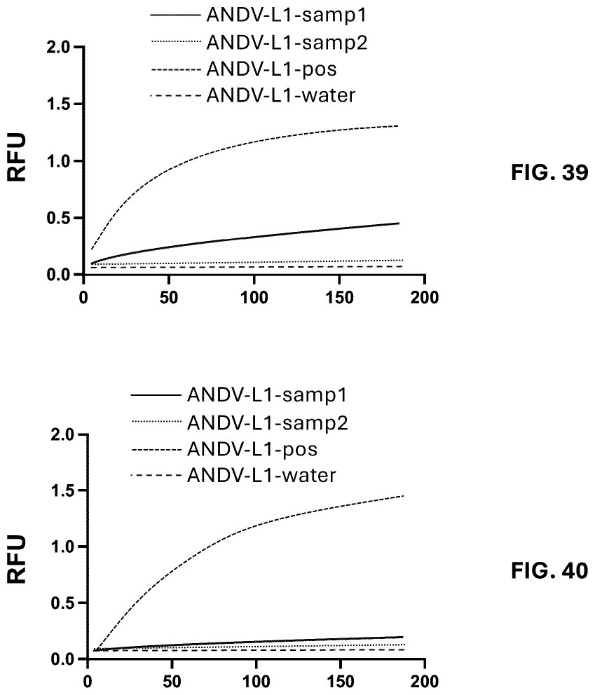

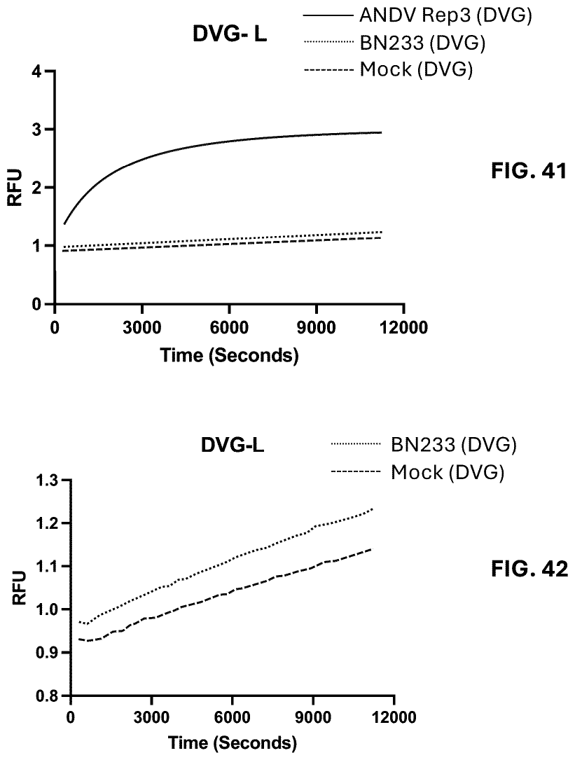

The disclosed approach can be used to detect and quantify ssRNA, as disclosed herein. Further, the disclosed approach was used to detect and quantify S-segment from two different types of hantavirus, ANDV (Andes Virus) (see FIG. 37) and TULV (Tula Virus) (see FIG. 38). The disclosed approach was also be used to detect and quantify L-segment from ANDV (see FIGS. 39 and 40). The disclosed approach was also be used to detect and quantify L-segment DVGs from ANDV (see FIGS. 41 and 42).

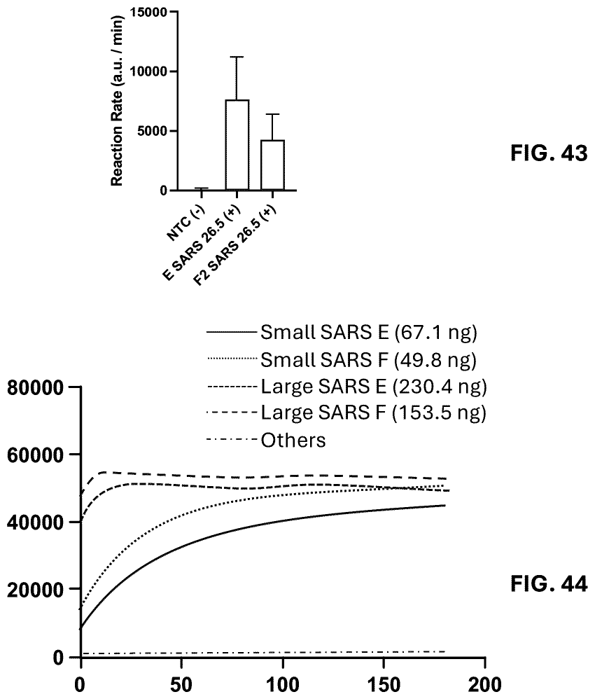

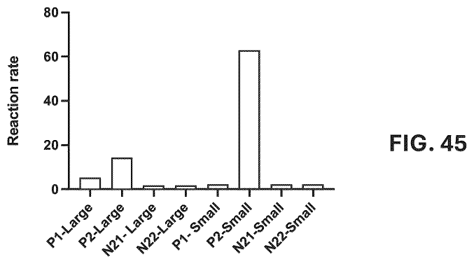

SARS-COV-2 also produces innate-immune stimulatory RNAs called small viral RNAs (svRNAs). The disclosed approach can detect 5′ ends from SARS-COV-2 infected samples (see FIGS. 43 and 44). In FIG. 43, detection of E SARS 26.5 and F2 SARS 26.5 were compared to no template control (NTC). In FIG. 44, small SARS E (67.1 ng), small SARS F (49.8 ng), small SARS C (28.7 ng), small SARS 1 (18.6 ng), large SARS E (230.4 ng), large SARS F (153.5 ng), large SARS C (33.4 ng), and large SARS 1 (14.4 ng) were detected; for simplicity, the SARS C and SARS 1 samples were listed as “Other”. As shown in FIG. 45, the disclosed approach can be used to detect SARS-COV-2 genome and svRNA.

What is claimed is:

1. A composition of matter, comprising:
a Cas13 enzyme;
a reporter;
a sequence-specific CRISPR RNA (crRNA); and
a buffer.

2. The composition of matter of claim 1, wherein the Cas13 enzyme is LbuCas13a or LwaCas13a.

3. The composition of matter of claim 1, wherein the Cas13 enzyme is a Cas13 enzyme other than LbuCas13a or LwaCas13a.

4. The composition of matter of claim 1, wherein the reporter is a quenched fluorescent reporter or an affinity-based reporter.

5. The composition of matter of claim 1, wherein the sequence-specific crRNA is configured to target a junction sequence formed upon aberrant viral RNA production.

6. The composition of matter of claim 5, wherein the aberrant viral RNA is mini viral RNA (mvRNA).

7. The composition of matter of claim 1, further comprising sample RNA.

8. A method for quantitative detection of a small, aberrant viral nucleic acid, comprising:
forming a test mixture by combining a detection mixture with a sample, the sample comprising an RNA sample, the detection mixture comprising:
a Cas13 enzyme;
a reporter;
a sequence-specific CRISPR RNA (crRNA); and
a buffer;
allowing a reaction within the test mixture to begin; and
determining a quantitative value representative of the reporter in the test mixture, where the quantitative value is a measure of an amount of target RNA present in the RNA sample.

9. The method of claim 8, wherein the reporter is a fluorescent reporter, a lateral flow reporter, a colorimetric reporter, or a luminescent reporter.

10. The method of claim 8, wherein the value is a fluorescence or lateral flow readout.

11. The method of claim 8, wherein the method is free of an amplification step.

12. The method of claim 8, further comprising pre-amplification of the RNA sample.

13. The method of claim 8, further comprising RNA fractionation.

14. The method of claim 8, wherein the small, aberrant viral nucleic acid comprises synthetic RNA.

15. The method of claim 8, wherein the small, aberrant viral nucleic acid comprises RNA produced by a virus.

16. The method of claim 15, wherein the virus is a coronavirus, an arenavirus, a hantavirus, a paramyxovirus, an Ebola virus, a picornavirus, or a flavivirus.

17. The method of claim 8, wherein the sample includes an antiviral, and the method further includes monitoring an infection status by repeating the forming, allowing, and determining steps and tracking the quantitative value over time.

18. A detection system, comprising:
a detection mixture container, where a composition of matter of claim 1 is disposed within the detection mixture container; and
a detector configured to detect fluorescence of the composition of matter of claim 1.

19. A detection kit, comprising:
a sample container; and
a detection mixture container, where a composition of matter of claim 1 is disposed within the detection mixture container.
